(19) United States
(12) Patent Application Publication
(10) Pub. No.: US 2005/0142259 A1
(43) Pub. Date: Jun. 30, 2005

(54) High pressure inactivation of pathogenic viruses

(76) Inventor: Richard S. Meyer, Tacoma, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/027,218

(22) Filed: Dec. 29, 2004

Related U.S. Application Data
(60) Provisional application No. 60/533,484, filed on Dec. 31, 2003.

(51) Int. Cl. G01N 33/02

(52) U.S. Cl. 426/335

(57) ABSTRACT

A process for inactivating pathogenic viruses in meat includes the steps of subjecting the meat to an elevated pressure for a predetermined period of time, adiabatically increase the temperature of the meat to a temperature not to exceed 50° C. while retaining the raw color meat characteristics.

### CROSS-REFERENCE TO RELATED APPLICATION

This application claims the benefit of U.S. Provisional Application No. 60/533,484, filed Dec. 31, 2003.

### FIELD OF THE INVENTION

The present invention relates to inactivation of pathogenic viruses and more particularly to the inactivation of pathogenic viruses in meat products.

### BACKGROUND OF THE INVENTION

Some meat products are contaminated with pathogenic viruses such as those that cause hoof and mouth disease. Before those meat products can be commercially distributed, the pathogenic viruses must be destroyed so as to avoid contamination of other meat products or injury to the consumer of those meat products. Pathogenic viruses are normally destroyed or inactivated by heating the meat product to an elevated temperature for a predetermined period of time until the viruses are effectively eliminated.

Heating the meat for the requisite time and temperature will cause at least partial and sometimes full cooking of the meat. As this occurs, the flavor, texture and color will be altered. Specifically, the color will turn, for example, from normal red or pink color for raw beef to a shade of gray. Even if the meat is only partially cooked, thus retaining much of its raw meat characteristics, the irreversible gray shading still occurs and is undesirable in a consumer product.

### SUMMARY OF THE INVENTION

In accordance with the present invention, applicants have discovered that pathogenic viruses in meat can be inactivated by introducing the meat into a pressure chamber and subjecting the meat to an elevated pressure for a period of time to adiabatically increase the temperature of the meat to an elevated level. This elevated temperature level should not exceed 50° C. This pressurization and concomitant heating eliminates pathogenic viruses while retaining raw colored meat characteristics. It is preferred that the minimum pressure to which the meat is raised is 200 MPa. It is more preferred that the meat temperature be at or below 0° C. before introduction into the pressure chamber. Thus, the meat before and after treatment in accordance with the present invention is maintained in a frozen state. The process of the present invention reduces the virus count by a 4 log count or higher, which is adequate to ship and distribute the meat in the consumer market.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a chart showing pressures and temperatures at which a 4 log count inactivation of viruses is achieved while retaining the optimum meat coloration.

### DETAILED DESCRIPTION OF THE PREFERRED INVENTION

The present invention is applicable to all kinds of meat including beef, pork, lamb and other types of meats normally consumed by individuals. The viruses can be inactivated in accordance with the present invention are those that typically contaminate meat products. An example of such a virus is that which causes hoof and mouth disease in other animals.

Virus inactivation in meat is effected by placing the meat in a pressure chamber and pressurizing the chamber to a predetermined pressure for a predetermined period of time. This causes an adiabatic increase in the temperature of the meat product. The combination of the pressurization and the temperature increase will inactivate, that is virtually destroy, any pathogenic virus present in the meat. A 4 log reduction in the pathogenic virus can be achieved in accordance with the present invention. This reduction meets FDA standards for elimination of pathogenic viruses in food products. A 4 log reduction means a virus count reduction on the order of at least 104 to 0 (or a count so low as to be immeasurable).

At the same time, the process of the present invention will not cause a significant change in the color of the meat especially if the adiabatic temperature increase does not exceed 50° C. The initial temperature of the meat can range from −40° to 45°, however, in the most preferred form of the invention it is preferred that the meat be frozen, that is less than 0° C. when subjected to the pressurization process of the present invention. It is preferred that the pressure be increased to a pressure of at least 200 MPa or preferably 250 MPa and most preferably 350 MPa. It is most preferable that the starting temperature of the meat be at 0° C., more preferably less than −10° C., and most preferably less than −20° C. At the same time, it is preferred that the final temperature not exceed 49° C. and preferably not exceed 35° C.

The pressure can be maintained for any period of time, however, it is preferable that the pressure be maintained for less than five minutes, preferably less than one minute and most preferably from five seconds to one minute. This latter range will effectively achieve a 4 log kill of pathogenic viruses.

The pressure can be applied to the meat in a single pulse or in multiple pulses. Multiple pulses may be more effective for certain kinds of viruses; however, it has been found that single pulse is effective for a virus such as that which causes hoof and mouth disease.

A wide variety of pressure vessels can be used. They are available from Flow International Corporation, Kent, Wash.; Mitsubishi Heavy Industries, Tokyo, Japan; Kobe Steel, Kobe, Japan; ABB Autoclave Systems Inc., Vasteras, Sweden; and Engineered Pressure Systems Inc., of Andover, Mass.

### EXAMPLE 1

A sample of beef contaminated with hoof and mouth disease virus is obtained, is tested and found to obtain 10³ to 10⁴ grams of virus per gram of meat. This level of virus is not acceptable for normal distribution in commercial channels. A 5 gram sample of the meat is placed in a plastic bag and the bag evacuated. The plastic bag is inserted in a pressure chamber. The bag is surrounded by an incompressible fluid such as water. A pressure pulse is then applied for one minute to the meat and then released. Similar meat samples having initial temperatures ranging from −40° C. to +40° C. are raised to pressures ranging from 100 MPa to 1400 MPa. The meat is then removed from the chamber and tested for the presence of live virus. In all cases, the live virus is virtually eliminated from all samples.

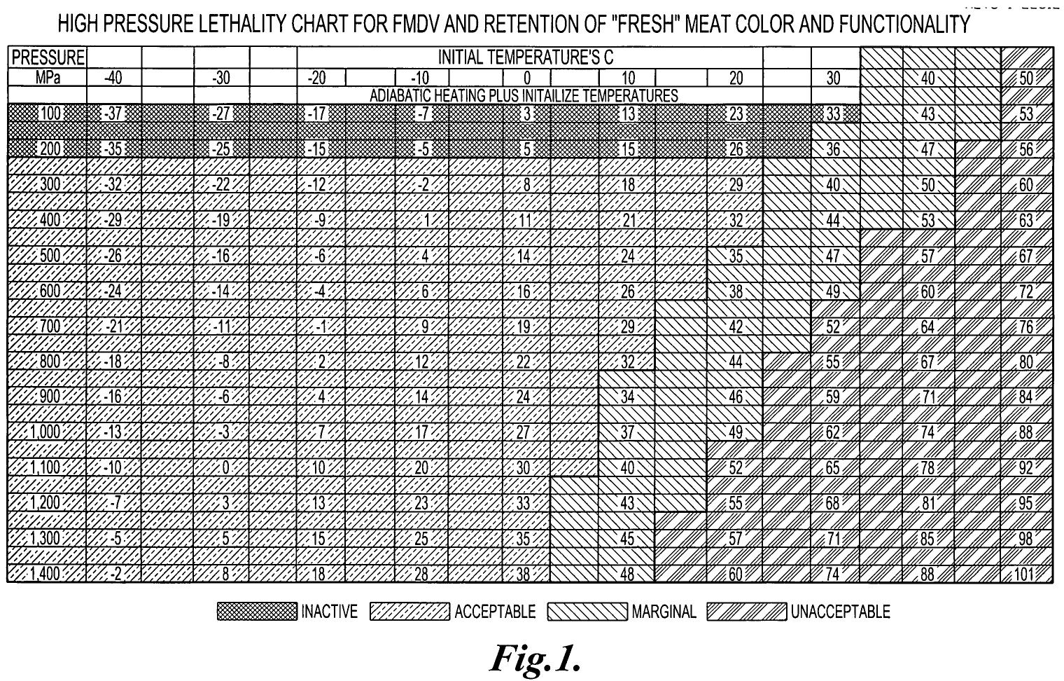

Each sample of meat is also measured for color and compared against its original raw meat color. FIG. 1 is a chart of pressure versus temperature on which the color change is overlaid. Viruses are not inactivated in the zone cross-hatched with the inactive (I) legend. If the sample color is not changed, the sample is labeled as acceptable (A); if the sample color is changed slightly but is still a commercially acceptable shade of pink, the sample is labeled as marginal (M); and if the fresh pink color of the sample is lost, the sample is labeled as unacceptable (U). It can be seen that acceptable color change is achieved by maintaining the initial temperature below 20° C. and raising the pressure to no more than 450 MPa. As the initial temperature decreases, the pressure can be raised higher without adversely affecting the coloration. In the same light, at a 20° C. initial temperature, an acceptable color can be achieved up to 450 MPa and a marginal color change can be achieved up to about 1000 MPa. However, when the pressure is raised above 1000 MPa with meat at an initial temperature of 20° C., the adiabatic temperature rise becomes too high and causes an unacceptable discoloration.

While the preferred embodiment of the invention has been illustrated and described, it will be appreciated that various changes can be made therein without departing from the spirit and scope of the invention.

1. A process for eliminating pathogenic viruses in meat, comprising:
introducing meat into a pressure chamber; and
subjecting the meat to an elevated pressure in the pressure chamber for a predetermined period of time to adiabatically increase the temperature of the meat to a level not to exceed 50° C. to eliminate pathogenic viruses while retaining raw-colored meat characteristics.

2. The process of claim 1, wherein the pressure is increased to at least 200 MPa.

3. The process of claim 2, wherein the pressure is at least 250 MPa.

4. The process of claim 3, wherein the pressure is at least 300 MPa.

5. The process of claim 1, wherein the temperature of the meat in the pressure chamber is initially at least 0° C.

6. The process of claim 5, wherein the meat has an initial temperature of at least −10° C.

7. The process of claim 6, wherein the meat has an initial temperature of at least −20° C.

8. The process of claim 2, wherein the elevated temperature is maintained for a period of at least 5 seconds.

9. The process of claim 8, wherein the elevated pressure is maintained for a period of from 5 seconds to 1 minute.

10. The process of claim 8, wherein the elevated temperature is maintained for a period of from about 5 seconds to about 5 minutes.

11. The process of claim 2, wherein the predetermined temperature level is no greater than 49° C.

12. The process of claim 11, wherein the predetermined temperature level is no greater than 35° C.

13. The process of claim 2, wherein a virus kill of at least 4 logs is obtained.
